(19) United States
(12) Patent Application Publication
(10) Pub. No.: US 2018/0163250 A1
(43) Pub. Date: Jun. 14, 2018

(54) Screening Broths Comprising Esculatin Compounds for the Detection of Specific Microorganisms

(71) Applicant: Neogen Corporation, Lansing, MI (US)

(72) Inventors: Christopher Andrew Potter, West Yorkshire (GB); Ezzeddine Fawaz Elmerhebi, Manchester (GB)

(21) Appl. No.: 15/835,647

(22) Filed: Dec. 8, 2017

Related U.S. Application Data
(60) Provisional application No. 62/432,121, filed on Dec. 9, 2016.

(51) Int. Cl. C12Q 1/42; G01N 21/78; G01N 21/80

(52) U.S. Cl. CPC C12Q 1/42; G01N 2333/31; G01N 21/80; G01N 21/78

(57) ABSTRACT

The present invention relates to the use of an esculatin compound in a method for detecting the presence or absence of an enzymatic reaction in a sample containing one or more enzymes and one or more first chromogenic indicator of one or more enzymatic reactions, wherein the esculatin compound is a compound of formula 1:

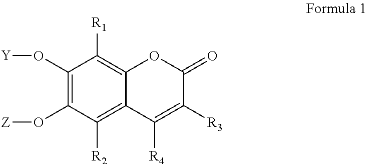

or a suitable salt or hydrate thereof.

### CROSS-REFERENCE TO RELATED APPLICATION

This U.S. Patent Application claims priority to U.S. Provisional Application: 62/432,121 filed on Dec. 9, 2016, the disclosure of which is considered part of the disclosure of this application and is hereby incorporated by reference in its entirety.

### TECHNICAL FIELD

The present invention relates to using esculatin compounds in a method of confirming enzymatic activity in the presence of another test. The invention further relates to using esculatin compounds in a method of masking a diagnostic test for enzymatic activity with a second, confirmatory test. The invention also relates to confirming the presence or absence of a specific type of bacteria in a sample.

### BACKGROUND

A wide range of synthetic analogues of naturally occurring substrates have been synthesized and used to detect a variety of microorganisms through the detection of specific enzymatic activity which produces chromogenic or fluorogenic substances when hydrolyzed from their partner molecule. The detection of specific activity can be carried out using non selective or culture media incorporating selective agents to which the target microorganisms are resistant and non-target organisms which would otherwise be growth competitors or display the same enzymatic activity as the target. One type of analogue substrate which can be used for this type of detection are derivatives of 3,4-cyclohexenoesculatin (CHE) which yield a metal binding molecule when target enzymatic activity liberates this moiety from the analogue substrate. In the presence of Iron this produces a distinct black coloration rather than a fluorogenic or colored product. This black compound can also be used as a “masking” reaction to allow the specific detection of target organisms where two or more enzymatic activities are present.

MRSA detection broths based on the principle of mannitol and trehalose utilization and cefoxitin were designed to be a selective system for high throughput screening of clinical MRSA samples. The broths allow high volume screening of the majority of patient samples by a low cost method with confirmation of the few presumptive positive samples by rapid methods. The broths achieved good performance relative to Mannitol salts broth enrichment and/or direct plating (>98% sensitivity and >96 selectivity). In order to further enhance the selectivity of this broth system, the use of a confirmatory enzymatic test targeted at the MRSA phosphatase activity was investigated. It was found that some indoxyl based chromogens did not produce sufficient chromophore product to detect this reaction in a liquid system and that at higher concentrations than that used in solid media demonstrated inhibitory or toxic effects. Fluorogenic phosphatase substrate, although giving a confirming reaction, suffered from the disadvantage of nonspecific fluorescence in the broth probably due to compound stability, and also necessitated the use of non-auto fluorescent glass or plastic tubes. A sample of CHE-phosphate was tested for activity in the MRSA screening broth, and unexpectedly produced a readily observable blackening reaction. Although not expected to give a significant reaction due to the assumed requirement for chromophore accumulation in colonies on agar plates for detection of enzymatic activity (as required for Indoxyl substrates), the intense blackening reaction was rapidly observed either when the substrate and ferric ammonium citrate was added to presumptive positive tubes at the end of incubation; or if CHE-phosphate was included in the broth prior to inoculation and incubation with Ferric ammonium citrate added to confirm presumptive positives after incubation. From this initial observation it was apparent that the CHE-chromogens could be successfully used in broth systems as well as solid plate media. CHE-chromogens also made possible a “masking” chromogen reaction in which more than one color or fluorogenic based end point detection of specific organism activity could be carried out in a single tube.

The dark brown/black precipitate formed when esculatin is reacted with ferric ammonium citrate has been used as an indicator for a variety of enzymatic reactions. Esculatin compounds have been shown to have general application as substrates for the detection of microorganisms, as disclosed in WO 1997/41138, and have been used in the specific identification of the presence of Salmonella in enterobacteria samples by detecting the activity of α-D-galactosidase, as disclosed in WO 1998/55644. Both WO 1997/41138 and WO 1998/55644 are incorporated herein by reference in their entirety.

### SUMMARY

The present invention includes a method for detecting the presence or absence of enzymatic activity from one or more microorganisms in a liquid sample that contains one or more first chromogenic indicator of the enzymatic activity, comprising the steps of:
a. contacting the liquid sample that contains the one or more first chromogenic indicator with an esculatin compound;
b. contacting the liquid sample that contains the one or more first chromogenic indicator and the esculatin compound with an iron containing compound; and
c. observing the liquid sample containing the one or more first chromogenic indicator, the esculatin compound, and the iron for the presence of a dark precipitate, wherein the dark precipitate can be seen in the presence of the one or more first chromogenic indicator,
   wherein the esculatin compound is a compound of formula 1:

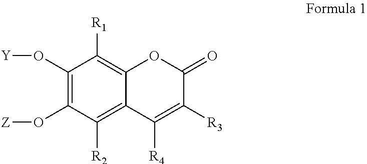

or a suitable salt or hydrate thereof, wherein

each of R₁, R₂, R₃, R₄, Y, and Z are defined herein.

The present invention can be used to achieve one or more, for example, 1, 2, or 3, diagnostic results in a single sample. The diagnostic results achieved by the present invention can include, but are not limited to color change, fluorescence, indoxyl based chromogenic reaction and masking CHE-chromogen reaction.

### DETAILED DESCRIPTION

### Embodiments of the Invention

In one aspect, the invention includes a method for detecting the presence or absence of enzymatic activity from one or more microorganisms in a liquid sample that contains one or more first chromogenic indicator of the enzymatic activity, comprising the steps of:
a. contacting the liquid sample that contains the one or more first chromogenic indicator with an esculatin compound;
b. contacting the liquid sample that contains the one or more first chromogenic indicator and the esculatin compound with an iron containing compound; and
c. observing the liquid sample containing the one or more first chromogenic indicator, the esculatin compound, and the iron for the presence of a dark precipitate, wherein the dark precipitate can be seen in the presence of the one or more first chromogenic indicator,
   wherein the esculatin compound is a compound of formula 1:

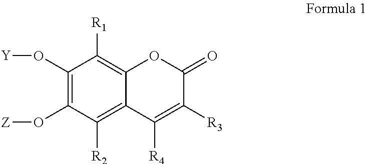

or a suitable salt or hydrate thereof, wherein

each of R₁ and R₂ is independently hydrogen, halogen, or another group which does not interfere with subsequent iron chelation;

each of R₃ and R₄ is independently hydrogen, (C₁-C₈) alkyl, (C₅-C₁₀) aryl-(C₁-C₈) alkyl, or a group of the general formula —CH₂(CH₂)ₙCOX, where n is a number from 0 to 3 and X represents a hydroxyl group, a carboxylic acid, an amino group, or another hydrophillic group; or

R₃ may alternatively represent an acyl group of the general formula —COR, in which R represents a (C₁-C₈)alkyl, (C₅-C₁₀) aryl-(C₁-C₈) alkyl, or (C₅-C₈) cycloalkyl group, provided that R₃ and R₄ between them contain at least three carbon atoms; or

R₃ and R₄ together with the carbon atoms to which they are attached form a (C₅-C₈) cycloalkene ring; and

each of Y and Z is independently hydrogen or an enzymatically cleavable group selected from the group consisting of
a phosphate group having the formula PO₃W₂ or PO₃V, wherein W is a sugar alcohol, hydrogen or a +1 metal cation and V is a +2 metal cation;
a —C(O)R₅ group, wherein R₅ is C₁₋₂₀ alkyl group; or
an α or β linked sugar residue,

provided that both Y and Z are not hydrogen.

In one embodiment of this aspect, the enzymatically cleavable group is an α or β linked sugar residue. In another embodiment, the enzymatically cleavable group is a β linked sugar residue. In a further embodiment, the sugar residue is selected from the group consisting of β-D-glucose, β-D-galactose, β-D-xylose, β-D-glycuronic acid, and N-acetyl-β-D-glucosamine. In still a further embodiment, the sugar residue is β-D-glucose.

In another embodiment of this aspect, the enzymatically cleavable group is a phosphate group having the formula PO₃W₂. In one embodiment, each W is independently hydrogen or a +1 metal cation selected from the group consisting of Li⁺, Na⁺, K⁺, Rb⁺, Cs⁺, Cu⁺, Ag⁺, Au⁺, and In⁺. In another embodiment, each W is independently hydrogen or a +1 metal cation selected from the group consisting of Li⁺, Na⁺, K⁺, Cs⁺, Ag⁺, and Au⁺. In another embodiment, each W is independently hydrogen or a +1 metal cation selected from the group consisting of Li⁺, Na⁺, and K⁺. In a further embodiment, each W is independently hydrogen or Na. In another further embodiment, each W is independently hydrogen or K.

In another embodiment, the enzymatically cleavable group is a phosphate group having the formula PO₃V. In one embodiment, V is a +2 metal cation selected from the group consisting of Be²⁺, Mg²⁺, Ca²⁺, Sr²⁺, Ba²⁺, Ti²⁺, Cr²⁺, Mn²⁺, Fe²⁺, Co²⁺, Ni²⁺, Cu²⁺, Zn²⁺, Cd²⁺, Hg₂²⁺, Hg²⁺, Sn²⁺, and Pb²⁺. In one embodiment, V is a +2 metal cation selected from the group consisting of Mg²⁺, Ca²⁺, Ba²⁺, Mn²⁺, Fe²⁺, Co²⁺, Ni²⁺, Cu²⁺, Zn²⁺, and Pb²⁺. In a further embodiment, V is a +2 metal cation selected from the group consisting of Mg²⁺, Ca²⁺, or Fe²⁺.

In another embodiment of this aspect, at least one W is a sugar alcohol. In one embodiment, the sugar alcohol is inositol. In a further embodiment, the inositol is the myo form of inositol.

In another embodiment, the enzymatically cleavable group is a —C(O)R₅ group, wherein R₅ is C₁₋₂₀ alkyl group. In one embodiment, R₅ is C₅₋₁₅ alkyl group. In another embodiment, R₅ is C₆₋₁₀ alkyl group. In a further embodiment, —C(O)R₅ has the structure

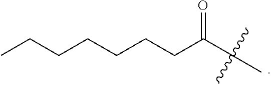 [SMILES: *C(=O)CCCCCCC]

In another further embodiment, —C(O)R₅ has the structure

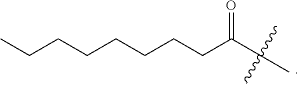 [SMILES: *C(=O)CCCCCCCC]

In one embodiment of this aspect, each of R₁ and R₂ is independently hydrogen, chloride, or bromide. In one embodiment, each of R₁ and R₂ is hydrogen.

In another embodiment of this aspect, each of R₃ and R₄ is independently hydrogen, (C₁-C₈) alkyl, (C₅-C₁₀) aryl-(C₁-C₈) alkyl. In one embodiment, each of R₃ and R₄ is independently (C₁-C₈) alkyl or (C₅-C₁₀) aryl-(C₁-C₈) alkyl. In a further embodiment, each of R₃ and R₄ is independently methyl, ethyl, propyl, isopropyl, n-butyl, isobutyl, tert-butyl, phenyl, or benzyl. In still a further embodiment, R₃ is n-butyl, phenyl, or benzyl and R₄ is methyl.

In another embodiment, one of R₃ and R₄ is hydrogen, and the other of R₃ and R₄ is (C₁-C₈) alkyl or (C₅-C₁₀) aryl-(C₁-C₈) alkyl. In a further embodiment, one of R₃ and R₄ is hydrogen, and the other of R₃ and R₄ is methyl, ethyl, propyl, isopropyl, n-butyl, isobutyl, tert-butyl, phenyl, or benzyl.

In one embodiment, R₃ and R₄ together with the carbon atoms to which they are attached form a (C₅-C₈) cycloalkene ring. In a further embodiment, R₃ and R₄ together with the carbon atoms to which they are attached form a cyclopentene, cyclohexene, or cycloheptene ring. In still a further embodiment, R₃ and R₄ together with the carbon atoms to which they are attached form a cyclohexene ring.

In one embodiment of this aspect, Y is hydrogen.

In another embodiment of this aspect, Z is hydrogen.

In some embodiments, the esculatin compound is selected from:

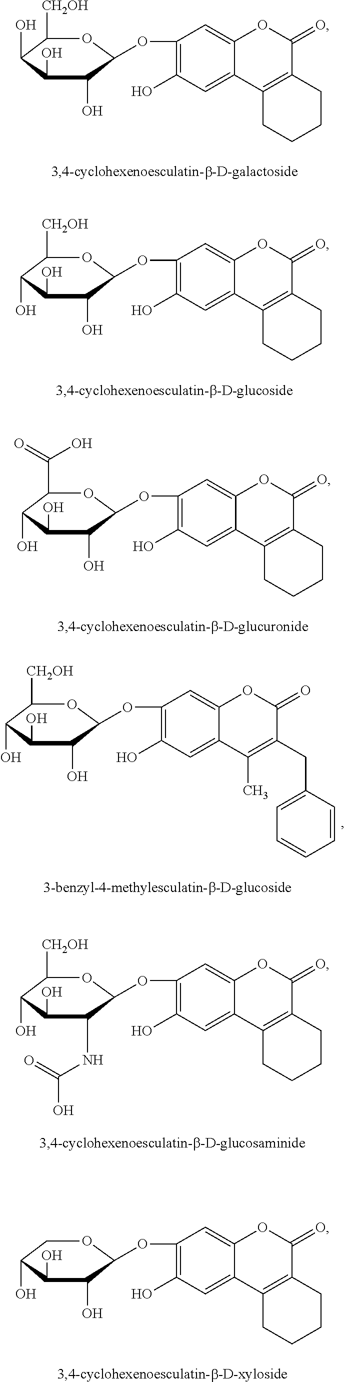

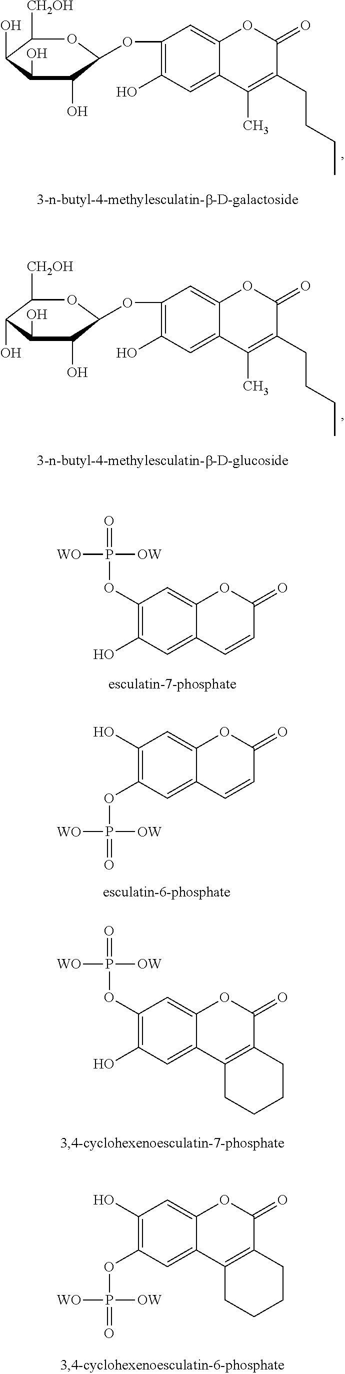

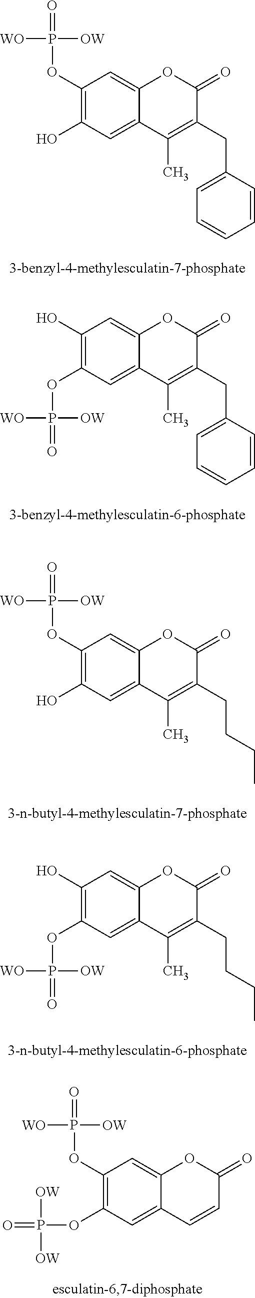

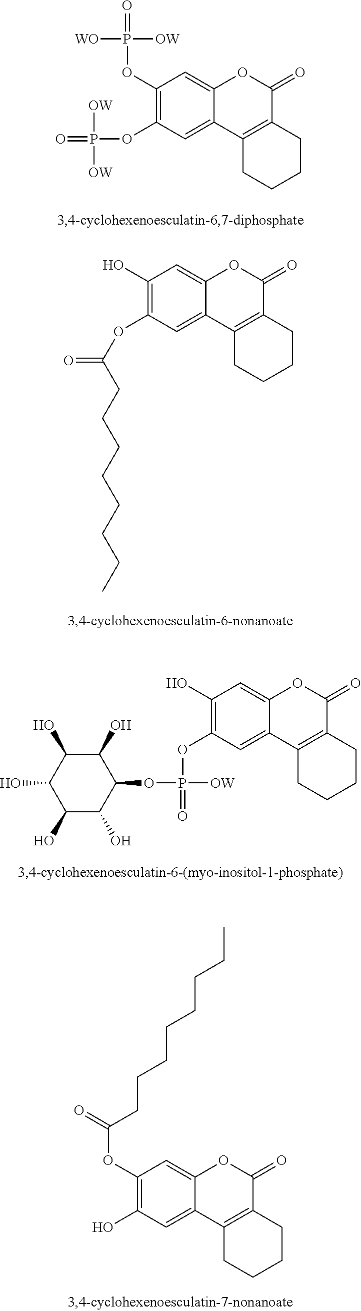

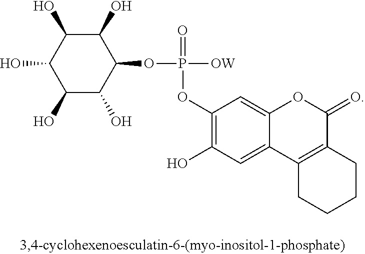

In a further embodiment, the esculatin compound is 3,4-cyclohexenoesculatin-6-phosphate

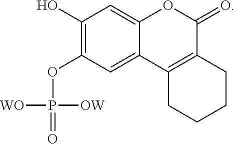

In one embodiment of this aspect, the one or more first chromogenic indicator of the enzymatic activity is a pH indicator. In a further embodiment, the pH indicator is selected from the group consisting of Cresol Red, Cresolphthalein (meta), Cresol Purple, Thymol Blue, Methyl Orange-Xylene Cyanol, Bromophenol Blue, Congo Red, Methyl Orange, Alizarin Red S, Bromocresol Green, Dichlorofluorescein, Methyl Red, Bromocresol Green/Methyl Red, Bromocresol Purple, Chlorophenol Red, Bromothymol Blue, Phenol Red, Naphtholphthalein (alpha), Phenolphthalein, Cresolphthalein (ortho), Thymolthalein, and Indigo Carmine, or combinations thereof.

In another embodiment, the one or more first chromogenic indicator of the enzymatic activity is a visible color indicator. In a further embodiment, the one or more first chromogenic indicator of the enzymatic activity is an indoxyl substrate. In another further embodiment, the one or more first chromogenic indicator of the enzymatic activity is 5-bromo-4-chloroindoxyl phosphate.

In one embodiment, the one or more first chromogenic indicator of the enzymatic activity is a fluorescent indicator. In another embodiment, the one or more first chromogenic indicator of the enzymatic activity is a 4-methylumbelliferyl substrate. In a further embodiment, the one or more first chromogenic indicator of the enzymatic activity is 4-methylumbelliferyl-β-D glucopyranoside. In another further embodiment, the one or more first chromogenic indicator of the enzymatic activity is 4-methylumbelliferyl-β-D glucuronide.

In one embodiment of this aspect, the dark precipitate masks the fluorescence or the color of the one or more first chromogenic indicator. In one embodiment, the presence or absence of the dark precipitate is a secondary or confirmatory test after the one or more first chromogenic indicator.

In another embodiment of this aspect, the one or more microorganisms is selected from a bacteria, a fungi, or a yeast. In one embodiment, the one or more microorganisms are bacterial microorganisms. In a further embodiment, the one or more bacterial microorganisms are selected from staphylococcus aureus, listeria, salmonella, clostridium, streptococcus, klebsiella, enterobacter, escherichia, citrobacter, proteus, bacillus, pseudomonas, lactobacillus, and coliforms. In still a further embodiment, the one or more bacterial microorganisms are clostridium microorganisms. In still a further embodiment, the clostridium microorganisms are clostridium perfringens.

In one embodiment, the staphylococcus aureus is methicillin resistant staphylococcus aureus. In one embodiment, the presence of the dark precipitate indicates that the microorganism is not methicillin resistant coagulase negative staphylococcus.

In one embodiment, the liquid sample is incubated prior to contacting with the esculatin compound. In one embodiment, the liquid sample is incubated for less than 48 hours prior to contacting with the esculatin compound. In a further embodiment, the liquid sample is incubated for about 22 hours prior to contacting with the esculatin compound.

In one embodiment, the liquid sample is incubated at a temperature from about 25° C. to about 45° C. In one embodiment, the liquid sample is incubated at a temperature from about 30° C. to about 45° C. In another embodiment, the liquid sample is incubated at a temperature from about 35° C. to about 40° C. In a further embodiment, the liquid sample is incubated at a temperature of about 37° C.

In one embodiment, the liquid sample is incubated after adding the esculatin and prior to adding the iron compound. In one embodiment, the liquid sample is incubated for less than 48 hours after adding the esculatin and prior to adding the iron compound. In a further embodiment, the liquid sample is incubated for about 22 hours after adding the esculatin and prior to adding the iron compound. In another embodiment, the liquid sample containing the esculatin is incubated at a temperature from about 25° C. to about 45° C. In another embodiment, the liquid sample is incubated at a temperature from about 30° C. to about 45° C. In a further embodiment, the liquid sample is incubated at a temperature from about 35° C. to about 40° C. In still a further embodiment, the liquid sample is incubated at a temperature of about 37° C.

In one embodiment, the liquid sample containing the iron compound is incubated. In one embodiment, the liquid sample containing the iron compound is incubated for less than 48 hours. In a further embodiment, the liquid sample containing the iron compound is incubated for about 22 hours. In one embodiment, the liquid sample containing the iron compound is incubated at a temperature from about 25° C. to about 45° C. In one embodiment, the liquid sample containing the iron compound is incubated at a temperature from about 30° C. to about 45° C. In another embodiment, the liquid sample containing the iron compound is incubated at a temperature from about 35° C. to about 40° C. In a further embodiment, the liquid sample containing the iron compound is incubated at a temperature of about 37° C.

In one embodiment, the iron containing compound comprises iron III. In a further embodiment, the iron containing compound is ferric ammonium citrate.

In one embodiment, the dark precipitate masks the one or more first chromogenic indicator. In a further embodiment, the method provides confirmation of an enzymatic reaction indicated by the one or more first chromogenic indicator. In another embodiment, the method provides further specificity of an enzymatic reaction indicated by the one or more first chromogenic indicator, when the one or more first chromogenic indicator indicates a plurality of possible enzymatic reactions.

In one embodiment, the one or more first chromogenic indicator in the liquid sample is two first chromogenic indicators of the enzymatic activity. In a further embodiment, the dark precipitate masks the two first chromogenic indicators of the enzymatic activity. In another embodiment, the one or more first chromogenic indicator is selected from colored indicators, fluorescent indicators, and combinations thereof.

In another embodiment, the two of the one or more first chromogenic indicator of the enzymatic activity are: one colored chromogenic indicator and one fluorescent indicator. In one embodiment, a colored indicator is an indoxyl substrate. In one embodiment, a fluorescent indicator is a 4-methylumbelliferyl substrate. In a further embodiment, the liquid sample contains 5-Bromo-4-chloro-3-indoxyl nonanoate as a colored first chromogenic indicator and 4-Methylumbelliferyl-β-D-glucuronide as a fluorescent first chromogenic indicator. In another further embodiment, the liquid sample contains 5-Bromo-4-chloro-3-indoxyl nonanoate as a colored first chromogenic indicator and 4-Methylumbelliferyl-β-D-glucoside as a fluorescent first chromogenic indicator.

In one embodiment of this aspect, the liquid sample comprises one or both of:
a. methicillin resistant staphylococcus aureus; and
b. methicillin resistant coagulase negative staphylococcus.

In one embodiment, the presence or absence of enzymatic activity is due to phosphatase activity from methicillin resistant staphylococcus aureus and/or methicillin resistant coagulase negative staphylococcus. In one embodiment, the compound of Formula 1 is 3,4-cyclohexenoesculatin-6-phosphate.

In one embodiment, the presence of a dark precipitate in the liquid sample is only seen in the presence of methicillin resistant staphylococcus aureus.

In another embodiment, the one or more first chromogenic indicator indicates the presence of methicillin resistant staphylococcus aureus or methicillin resistant coagulase negative staphylococcus by color change. In another embodiment, the one or more first chromogenic media indicates the presence of methicillin resistant staphylococcus aureus or methicillin resistant coagulase negative staphylococcus by color change prior to adding the iron compound to the liquid sample. In one embodiment, the one or more first chromogenic indicator indicates the presence of methicillin resistant staphylococcus aureus or methicillin resistant coagulase negative staphylococcus by color change prior to adding the esculatin compound to the liquid sample.

In one embodiment, the presence of a dark precipitate in the liquid sample containing the iron compound masks the one or more first chromogenic indicator and confirms that the staphylococcus aureus indicated by the first chromogenic media is Methicillin resistant staphylococcus aureus (MRSA). In another embodiment, the absence of a dark precipitate in the liquid sample containing the iron compound confirms that the staphylococcus aureus indicated by the one or more first chromogenic media is methicillin resistant coagulase negative staphylococcus.

In one embodiment, the liquid sample is a broth. In a further embodiment, the broth is a MRSA broth sold by Lab M limited under the trade name LAB588™.

In one embodiment, the liquid sample comprises listeria. In a further embodiment, the enzymatic activity is due to listeria. In still a further embodiment, the compound of Formula 1 is 3,4-cyclohexenoesculatin-β-D-glucoside.

In one embodiment, the one or more first chromogenic indicator indicates the presence of listeria by color change prior to adding the iron compound to the liquid sample. In another embodiment, the one or more first chromogenic indicator indicates the presence of listeria by color change prior to adding the esculatin compound to the liquid sample. In another embodiment, the presence of a dark precipitate in the liquid sample containing the iron compound masks the one or more first chromogenic indicator and confirms the presence of listeria.

In one embodiment, the one or more first chromogenic indicator is a fluorogenic analog substrate. In a further embodiment, the first chromogenic indicator is 4-methylumbelliferyl-β-D glucoside. In a further embodiment, the one or more first chromogenic indicator is 4-methylumbelliferyl-β-D glucuronide.

In one embodiment, the liquid sample is a broth. In a further embodiment, the broth is a Listeria broth sold by Lab M limited under the trade name LAB589™. In another further embodiment, the broth is a Brain heart infusion broth sold by Lab M limited under the trade name LAB049™.

Definitions

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As used herein, the term “esculatin compound” refers to any compound having an esculatin core structure and an enzymatically cleavable group. Preferred esculatin compounds are those where exposure to an iron containing compound such as ferric ammonium citrate (FAC) produces no chemical reaction until the enzymatically cleavable group is removed.

As used herein, the term “iron containing compound” refers to any compound having a bound iron atom, and will produce a dark precipitate (dark color) when exposed to esculatin. A preferred iron containing compound is ferric ammonium citrate.

As used herein, the term CHE-phosphate refers to the compound sodium 3,4-cyclohexenoesculatin-6-phosphate (sodium 3-hydroxy-6-oxo-7,8,9,10-tetrahydro-6H-benzo[c]chromen-2-yl phosphate), which has the structure:

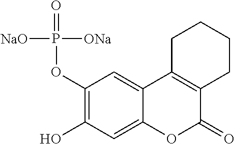

As used herein, the terms “ferric ammonium citrate” and “Ammonium ferric citrate” have identical meanings and refer to an iron containing compound having the molecular formula (NH₄)₅Fe(C₆H₄O₇)₂.

As used herein, the term “broth” refers to any liquid media that contains bacterial nourishment, and can be used in the proliferation of bacterial cells. In some embodiments, a broth relies on fermentation of sugar to produce acid to indicate presence of metabolic activity. In some further embodiments, the broth employs a pH or redox dye to detect metabolic activity through a change in pH. Some examples of broths are, but not limited to, MRSA ColorScreen™, Enterobacteriaceae Enrichment broth, MacConkey broth, Brilliant green bile broth, Methyl Red-Voges Proskauer broth and phenol red broth (sugar fermentation broth). An example of a fluorogenic broth is, but not limited to, LAB077 ColorScreen™, a modified lauryl sulphate tryptose broth with MUG and tryptophan. Some examples of commercially available broths (Sigma Aldrich) are, but not limited to, BRILA MUG Broth for microbiology, HiCrome™ Rapid Coliform Broth, LST-MUG Broth, M1678 MUG EC broth, 4-Methylumbelliferyl β-D-Glucuronide Escherichia coli Broth, Fluorocult LMX broth, Readycult coliforms, ColiLert, Coliquick, Colisure

Another preferred broth described herein is the LAB588 broth, supplied by Lab M limited.

As used herein, an “alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylarnino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic-SO₂-], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as halo, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, heterocycloaliphaticamino, or aliphaticsulfonylamino], sulfonyl [e.g., alkyl-SO₂—, cycloaliphatic-SO₂—, or aryl-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphatic-SO₂— or amino-SO₂—]; sulfinyl [e.g., aliphatic-S(O)— or cycloaliphatic-S(O)—]; sulfanyl [e.g., aliphatic-S—]; cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl [e.g., mono-, di (such as p,m-dihaloaryl), and (trihalo)aryl]; (carboxy)aryl [e.g., (alkoxycarbonyl)aryl, ((aralkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl or ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl. A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl. A cycloalkyl or cycloalkenyl group can be optionally substituted with one or more substituents such as aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl [e.g., alkyl-SO₂— and aryl-SO₂—], sulfinyl [e.g., alkyl-S(O)—], sulfanyl [e.g., alkyl-S—], sulfoxy, urea, thiourea, sulfamoyl, sulfamide, oxo, or carbamoyl.

As used herein, an “acyl” group refers to a formyl group or R^{X}—C(O)—(such as alkyl-C(O)—, also referred to as “alkylcarbonyl”) wherein each of R^{X} and R^{Y} is independently hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. Acetyl and pivaloyl are examples of acyl groups.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

As used herein the term “hydrophilic” refers to a chemical or material having a tendency to mix with, dissolve in, or be wetted by water.

As used herein the term “hydrophobic” refers to a chemical or material having a tendency to repel or fail to mix with water.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, (Z) and (E) conformational isomers, and tautomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

Materials and Methods

In one aspect of the present invention, the esculatin compounds are used as a diagnostic indicator of enzymatic activity. Preferably, the esculatin compound comprises an enzymatically cleavable group as provided in Scheme 1 below. Accordingly, while the esculatin compound alone can form the dark colored complex with ferric ammonium citrate, no complex is formed while the enzymatically cleavable group is still attached.

In a further aspect of the present invention, a broth is provided to culture a bacterial sample, which further contains an initial test (e.g., with one or more first chromogenic indicator) for the enzymatic activity of the bacteria. Addition of the esculatin compounds of the invention, followed by the addition of ferric ammonium citrate provides a secondary test for the enzymatic activity of the bacteria by the presence or absence of a dark precipitate, in accordance with Scheme 1. In some embodiments, the dark precipitate formed as a result of the secondary test involving the esculatin compound masks the initial test provided in the broth. It shall be understood that the present invention can be practiced in various types of bacterial growth media, such as agar, and is not limited to the broth media disclosed herein.

In one aspect of the present invention, the enzymatically cleavable group can be any chemical group that can be removed by the activity of a specific enzyme. After removal of the cleavable group by a specific enzyme, addition of ferric ammonium citrate will provide a dark precipitate, giving a positive indication of the presence of the enzyme.

All incubations of samples disclosed herein were performed with a LEEC P3 incubator. All florescence observations and measurements were performed with a Vilber Lourmat TFX 20 h transiluminator.

All esculatin compounds described in the examples provided herein, with the exception of 3,4-cyclohexenoesculatin diphosphate, were specially synthesized by the commercial supplier BioSynth AG (Rietlistr 4, 9422 Staad, Switzerland) or Glycosynth Limited (14 Craven Court, Winwick Quay, Warrington, Cheshire. WA2 8QU England). 5-bromo-4-chloroindoxyl phosphate was provided by Gee Lawson (1st Floor, Premier House, 309 Ballards Lane, North Finchley, London N12 8LY—England), and 4-methylumbelliferyl-β-D glucoside (MC406) was provided by Inalco (S.P.A) Via Arcivescovo Calabiana, 18, 20139 Milano, Italy. 4-methylumbelliferyl-β-D glucopyranoside (M6336) was provided by the Sigma Aldrich Company Ltd, The Old Brickyard, New Road Gillingham, Dorset SP8 4XT United Kingdom.

All broths used for biological growth were provided by LAB M Limited (Topley House, 52 Wash Lane, Bury, Lancashire BL9 6AS, United Kingdom), as further noted in the Examples.

### EXAMPLES

### Example 1

### Synthesis of 3,4-Cyclohexenoesculatin Diphosphate

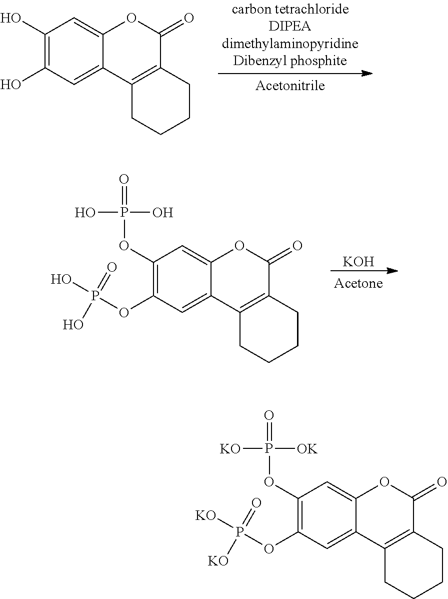

Cyclohexenoesculatin (1.16 g, 5.0mmol) was dissolved in dry acetonitrile (30 ml) in a three neck round bottomed flask with magnetic stirrer bar and fitted with a thermometer and nitrogen inlet port. The stirred solution was cooled in an ice/salt bath to −12° C. and charged with carbon tetrachloride (7.5 g), DIPEA (2.6 g), and dimethylaminopyridine (0.13 g). Dibenzyl phosphite (4.0 g) in acetonitrile (4 ml) was slowly added so as to keep the temperature effectively constant. The reaction was continued at low temperature for 30-40 minutes, after which time TLC analysis revealed almost total conversion to a fast running spot (the tetrabenzyl ester). The reaction was quenched by the addition of a pH 4 phosphate buffer (20 ml) and the reaction mixture extracted with propyl acetate (50 ml). The organic layer was washed twice with brine and dried with anhydrous magnesium sulfate. Rotary evaporation of the extract produced a viscous light colored oil, which crystallized on long standing.

Hydrogenolysis

The oil from the previous step was dissolved in dry tetrahydrofuran (25 ml) and treated with 10% palladium on carbon. A stream of hydrogen was passed through in an interrupted manner. The progress was followed by TLC and showed the progressive appearance of a baseline product, which reacted as an acid on addition of indicator. When the hydrogenolysis was complete, the catalyst was removed by filtration and washed with THF. The combined THF solutions were evaporated under reduced pressure and the product dissolved in acetone (˜10 ml). A concentrated solution of potassium hydroxide in butan-1-ol was added until the solution was at pH 9. The solution was cooled and 3,4-cyclohexenoesculatin diphosphate tetra-potassium salt crystallized out of solution upon standing. The white semi-crystalline product was removed by suction filtration, washed with methanol and dried.

Alternatively, the free acid could be dissolved in ethanol and the pH adjusted with concentrated aqueous KOH to pH 9 to obtain an identical product.

### Example 2

### CHE-Phosphate Compounds to Detect Phosphatase Activity in a MRSA Screening Broth

CHE-phosphate, and 3,4-cyclohexenoesculatin diphosphate were dissolved at 200 mg/ml in a 50:50 mixture of dimethylformamide and water. 5-bromo-4-chloroindoxyl phosphate was dissolved at 200 mg/ml in a 50:50 mixture of ethanol and water. The mixtures of compounds were added individually to a MRSA broth (LAB588; Lab M limited) pre-dispensed to 10 ml volumes, after supplementation with cefoxitin selective agent, as 25 μL additions from the prepared stock solutions.

A 0.5 Macfarlane standard suspension of methicillin resistant staphylococcus aureus strains were prepared from 18-24 h cultures grown in tryptic soy broth (LAB004; Lab M Limited) at 37° C. These were diluted and added to the MRSA broth samples in an amount sufficient to inoculate each sample with 100-1000 colony forming units (cfu). Target concentration was achieved by diluting a culture to a target level and inoculating sets of tubes at different inoculum dilutions so as to achieve the target range. The actual quantity inoculated was confirmed by plating 50 μl of the dilution onto Tryptone soy agar (LAB011) and incubating the plates for 18-24 h at 37° C. and counting the resulting colonies. This information was used to determine which tubes had been inoculated within the target dosing range.

After incubation, the samples were observed for visual growth and a color change due to acid production from mannitol and trehalose. The broth is designed to selectively indicate a positive presence of MRSA by a yellow color, or a presumptive positive presence of MRSA by an orange color (Table 1). To the CHE-phosphate and 3,4-cyclohexenoesculatin diphosphate was added 50 μL of a 250/mg/ml stock solution of Ferric ammonium citrate and samples were allowed to incubate at ambient temperature for 5 minutes. After this time samples were observed for blackening indicating the presence of free CHE and phosphatase activity (Table 2).

TABLE 1
Initial color change indicating the presence of MRSA

| MRSA Strain | Monophosphate | Diphosphate | Indoxyl phosphate alone |
|---|---|---|---|
| MRSA 3 | Growth (yellow) | Growth (yellow) | Growth (orange) |
| MRSA 15 | Growth (yellow) | Growth (yellow) | Growth (orange) |
| S. aureus NCTC 12493 | Growth (yellow) | Growth (yellow) | Growth (orange) |
| MRSA 15A (clinical isolate) | Growth (yellow) | Growth (yellow) | Growth (orange) |
| MRSA 15B (clinical isolate) | Growth (yellow) | Growth (yellow) | Growth (orange) |
| MRSA 15C (clinical isolate) | Growth (orange) | Growth (yellow) | Growth (orange) |
| MRSA 16A (clinical isolate) | Growth (orange) | Growth (yellow) | Growth (orange) |
| MRSA 16B (clinical isolate) | Growth (orange) | Growth (yellow) | Growth (orange) |
| MRSA 16C (clinical isolate) | Growth (orange) | Growth (yellow) | Growth (orange) |
| Uninoculated control | no growth red | no growth red | no growth red |

TABLE 2
blackening after the addition of ferric ammonium citrate
indicating the presence of free 3,4-cyclohexenoesculatin

| MRSA Strain | Monophosphate | Diphosphate |
|---|---|---|
| MRSA 3 | Blackening | No change |
| MRSA 15 | Blackening | No change |
| S. aureus NCTC 12493 | Blackening | No change |
| MRSA 15A (clinical isolate) | Blackening | No change |
| MRSA 15B (clinical isolate) | Blackening | No change |
| MRSA 15 C (clinical isolate) | Blackening | No change |
| MRSA 16A (clinical isolate) | Blackening | No change |
| MRSA 16B (clinical isolate) | Blackening | No change |
| MRSA 16 C (clinical isolate) | Blackening | No change |
| Uninoculated control | No change | No change |

Table 1 demonstrates that CHE-phosphate and 3,4-cyclohexenoesculatin diphosphate are not inhibitory for the growth of MRSA. Further, enzymatic phosphatase activity is specific for CHE-phosphate (Table 2). In addition, no detectable color or masking reaction (blackening reaction) was obtained for the equivalent Indoxyl substrate (monophosphate).

### Example 3

### Use of CHE-Phosphate to Discriminate Between Methicillin Resistant Staphylococcus aureus and Methicillin Resistant Coagulase Negative Staphylococcus

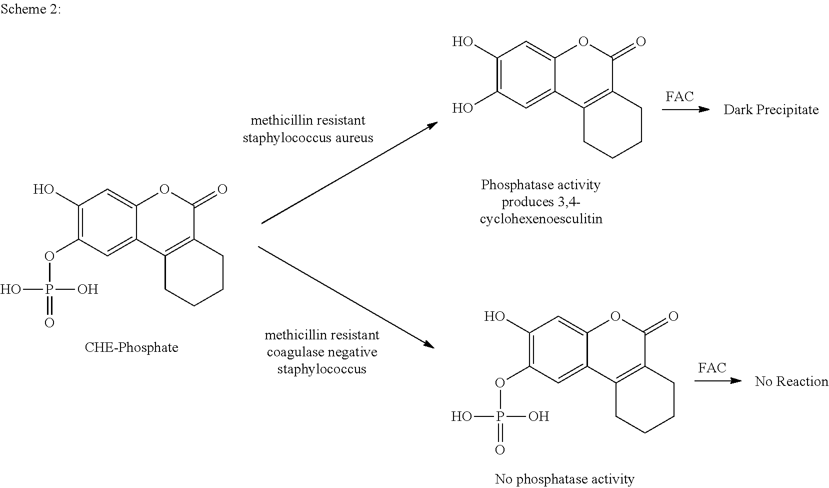 [SMILES: O=c1oc2cc(O)c(O)cc2c2c1CCCC2 ; O=c1oc2cc(O)c(OP(=O)(O)O)cc2c2c1CCCC2 ; O=c1oc2cc(O)c(OP(=O)(O)O)cc2c2c1CCCC2]

Scheme 2 above provides a pictorial representation of CHE-phosphate discriminating between methicillin resistant staphylococcus aureus and methicillin resistant coagulase negative Staphylococcus. The single phosphate group from CHE-phosphate is removed by the phosphatase activity of methicillin resistant staphylococcus aureus, but not by the coagulase negative staphylococcus, which lack this phosphatase activity. Addition of ferric ammonium citrate produces the dark, insoluble iron-3,4-cyclohexenoesculatin complex indicating the presence of methicillin resistant staphylococcus aureus. The dark complex is not formed between ferric ammonium citrate and CHE-phosphate, and therefore indicates the absence of phosphatase activity. Accordingly, a lack of a dark precipitate indicates the absence of methicillin resistant staphylococcus aureus. CHE-diphosphate does not produce the same dark precipitate, and therefore this activity is specific for CHE-monophosphate.

A panel of MRSA strains and coagulase negative staphylococci (clinical isolates) were cultured and tested in MRSA broth with CHE-phosphate using the same methodology as Example 2 CHE-phosphate was Dissolved at 200 mg/ml in 50:50 mixture of dimethylformamide and water. LAB588 (MRSA broth, Lab M limited) pre-dispensed to 10 ml volumes was supplemented with 25 μL aliquots from the prepared stock solution. A 0.5 Macfarlane standard suspension of methicillin resistant staphylococcus aureus and coagulase negative staphylococci strains were prepared from 18-24 h cultures grown in tryptic soy broth (LAB004, Lab M Limited) at 37° C. These were diluted to an amount sufficient to inoculate each sample with 100-1000 cfu. Samples were then incubated at 37° C. for 22 hours and observed for growth and color change. 50 μL aliquots of a 250 mg/ml stock solution of Ferric ammonium citrate (FAC) was then added to each sample and allowed to incubate at ambient temperature for 5 minutes. The samples were then observed for blackening indicating phosphatase activity by the presence of free 3,4-cyclohexenoesculatin (Table 3).

TABLE 3
CHE-phosphate discriminates between methicillin
resistant staphylococcus aureus and methicillin
resistant coagulase negative staphylococcus

| Strain | Before addition FAC | After addition of FAC |
|---|---|---|
| MRSA 15 | Yellow | Black |
| S. aureus NCTC 12493 | Yellow | Black |
| MRSA 3 | Yellow | Black |
| MRSA 75 | Orange | Black |
| MRSA 16A | Orange | Black |
| MRSA 16B | Orange | Black |
| CNS 22 | Orange | Orange |
| CNS 28 | yellow | yellow |
| CNS 33 | orange | orange |
| Uninoculated control | Red | Red |

As provided by Table 3, CHE-phosphate is specific for the phosphatase enzyme of MRSA, which is absent in the methicillin resistant coagulase negative staphylococci. Further, the presumptive positive reactions due to indicative color change in the broth could be confirmed through the use of the 3,4-cyclohexenoesculatin chromogen.

### Example 4

### 3,4-Cyclohexenoesculatin Glucoside for Pre-incubation and Post-Incubation Detection of Enzymatic Activity in a Broth System

3,4-cyclohexenoesculatin glucoside, was dissolved at 200 mg/ml in 50:50 mixture of dimethylformamide and water. The mixture was then added to pre-dispensed 10 ml volume samples of Listeria express broth (LAB589 from Lab M limited) as 25 μL additions from the prepared stock solutions. Additional broth samples without added chromogen were prepared and inoculated at the same time as the chromogen containing samples. The stock solution was then stored at −20° C. for 24 hours. A 0.5 Macfarlane standard suspension of Listeria monocytogenes, NCTC5348 and NCTC10527, was prepared from 18-24 hour cultures grown in tryptic soy broth (LAB004 from Lab M Limited) at 37° C. These were diluted so as to inoculate each sample with 100-1000 or 10,000-100,000 cfu. Samples were then incubated at 37° C. for 24 hours. The samples were then observed, and bacterial growth confirmed. 50 μL of a 250/mg/ml stock solution of ferric ammonium citrate was then added to each sample. Samples without 3,4-cyclohexenoesculatin glucoside chromogen were then supplemented with 25 μL additions from the stored 3,4-cyclohexenoesculatin glucoside stock solution and observed at ambient temperature for 30 minutes.

A strong black reaction occurred instantly on the addition of ferric ammonium citrate in the samples that were incubated with 3,4-cyclohexenoesculatin glucoside. In the samples having 3,4-cyclohexenoesculatin glucoside added post incubation, the strong black reaction was observed after about 15 minutes (Table 4).

TABLE 4
detection of enzymatic activity by 3,4-cyclohexenoesculatin glucoside

| | 3,4-cyclohexeno- | t = 0 minutes | t = 15 minutes |
|---|---|---|---|
| | esculatin glucoside | post iron addition | post iron addition |
| L. monocytogenes NCTC | pre incubation | Black | Black |
| 5348 100 cfu | | | |
| L. monocytogenes NCTC | pre incubation | Black | Black |
| 5348 10000 cfu | | | |
| Lmonocytogenes NCTC | pre incubation | Black | Black |
| 10527 100 cfu | | | |
| Lmonocytogenes NCTC | pre incubation | Black | Black |
| 10527 10000 cfu | | | |
| L. monocytogenes NCTC | post incubation | yellow | Black |
| 5348 100 cfu | | | |
| L. monocytogenes NCTC | post incubation | yellow | Black |
| 5348 10000 cfu | | | |
| Lmonocytogenes NCTC | post incubation | yellow | Black |
| 10527 100 cfu | | | |
| Lmonocytogenes NCTC | post incubation | yellow | Black |
| 10527 10000 cfu | | | |

### Example 5

### Use of 3,4-Cyclohexenoesculatin Glucoside to Mask Fluorogenic Reactions

4-methylumbelliferyl-β-D glucopyranoside (MC633 from Sigma Aldrich) was dissolved in water at 100 mg/ml and 25 μL of this stock solution added to pre-dispensed 10 mL volumes of Lab589 (Lab M Limited) and Lab049 (Brain heart infusion broth Lab M limited). Listeria monocytogenes NCTC 10257 were prepared from 18-24 hour cultures grown in tryptic soy broth (LAB004 from Lab M Limited) at 37° C. These were diluted so as to inoculate each sample with 100-1000 cfu. Samples were then incubated at 37° C. for 24 hours. Samples were then observed and confirmed for bacterial growth, and further observed under an ultra violet light for fluorescence. The samples were then supplemented with 3,4-cyclohexenoesculatin glucoside (Biosynth C9218), from a 200 mg/ml in 50:50 mixture of dimethylformamide and water. 50 μL of a 250/mg/ml stock solution of ferric ammonium citrate was added to each sample, observing visually for blackening and under ultra violet light for fluorescence masking after 15 minutes incubation at ambient. Table 5 provides that 3,4-cyclohexenoesculatin glucoside can be used as an additional masking reagent to confirm fluorogenic based broth systems.

TABLE 5

| | |
|---|---|
| t = 0 minutes post | T = 15 minutes post |
| iron addition | iron addition |

LAb589 broth

| | | |
|---|---|---|
| 4-methylumbelliferyl-β-D | fluorescent under uv light | fluorescent under uv light |
| glucopyranoside | | |
| 4-methylumbelliferyl-β-D | fluorescent under uv light | Black not fluorescent under uv |
| glucopyranoside + CHE glucoside | | |

LAB049 broth

| | | |
|---|---|---|
| 4-methylumbelliferyl-β-D | fluorescent under uv light | fluorescent under uv light |
| glucopyranoside | | |
| 4-methylumbelliferyl-β-D | fluorescent under uv light | Black not fluorescent under uv |
| glucopyranoside + CHE glucoside | | |

### Example 6

### Use of 3,4-Cyclohexenoesculetinephosphate Disodium Salt to Mask Indoxyl and Fluorogenic Reactions

4-Methylumbelliferyl-β-D-glucuronide (MUG) (LAB406 from LAB M Ltd.) was dissolved in dimethylformamide (DMF) at 80 mg/mL. 0.5 mL of this stock solution was added to 200 mL of pre-prepared Buffered Peptone Water “BTW” (LAB204 from Lab M Ltd), so that the final concentration of MUG in the media was 0.2 g/L. Escherichia coli ATCC 25922, Pseudomonas aeruginosa ATCC 27853 and Staphylococcus aureus ATCC 25923 were separately grown for 18-24 hours at 37° C.±1° C. in Tryptone Soy Broth (LAB004, Lab M Ltd). Serial dilutions of these cultures were made in maximum recovery diluent (LAB103, Lab M Ltd) so that 50 μL contained 100-1000 CFU. 50 μL of all three cultures were added to 10 mL of the previously described BPW+MUG. This was then incubated 18-24 hours at 37° C.±1° C. 200 μL of this enrichment was then added to three separate microplate wells. No addition was made to the first well. 20 μL of a 10 mg/mL solution of 5-Bromo-4-chloro-3-indoxyl nonanoate “X” (70089, Glycosynth Ltd) dissolved in DMF was added to the second and third well. 50 μL of a 50 mg/mL solution of 3,4-cyclohexenoesculetinephosphate disodium salt (C-9216, Biosynth AG) dissolved in DMF, as well as 50 μL of a 125 mg/mL solution of a filter sterilized (0.2 μm) Ferric ammonium citrate solution dissolved in water was added to third well only. The microplate was then incubated at 37° C.±1° C. for 90 minutes. Table 6 provides that 3,4-cyclohexenoesculetinephosphate disodium salt can be used as an additional masking reagent to confirm both indoxyl chromogen and fluorogenic broth based systems. In this test the microorganisms were tested in a single mixed culture sample. The result given is for the mixed samples.

TABLE 6

| | Visible | Appearance under |
|---|---|---|
| | appearance/color | 365 nm light (UV) |
| Well 1 (BPW + MUG) | Turbid, no color | Fluorescent |
| Well 2 (BPW + MUG + | Blue | Fluorescent |
| X-nonanoate) | | |
| Well 3 (BPW + MUG + | Black | Masked |
| X-nonanoate + CHE-phosphate) | | Fluorescence |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims.

What is claimed is:

1. A method for detecting the presence or absence of enzymatic activity from one or more microorganism in a liquid sample that contains one or more first chromogenic indicator of the enzymatic activity, comprising the steps of:
a. contacting the liquid sample that contains the one or more first chromogenic indicator with an esculatin compound;
b. contacting the liquid sample that contains the one or more first chromogenic indicator and the esculatin compound with an iron containing compound; and
c. observing the liquid sample containing the one or more first chromogenic indicator, the esculatin compound, and the iron for the presence of a dark precipitate, wherein the dark precipitate can be seen in the presence of the one or more first chromogenic indicator,
wherein the esculatin compound is a compound of formula 1: 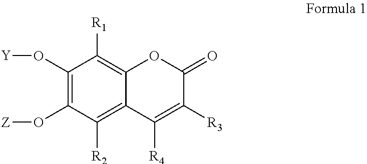
or a suitable salt or hydrate thereof, wherein
each of R₁ and R₂ is independently hydrogen, halogen, or another group which does not interfere with subsequent iron chelation;
each of R₃ and R₄ is independently hydrogen, (C₁-C₈) alkyl, (C₅-C₁₀) aryl-(C₁-C₈) alkyl, or a group of the general formula —CH₂(CH₂)ₙCOX, where n is a number from 0 to 3 and X represents a hydroxyl group, a carboxylic acid, an amino group, or another hydrophillic group; or
R₃ may alternatively represent an acyl group of the general formula —COR, in which R represents a (C₁-C₈)alkyl, (C₅-C₁₀) aryl-(C₁-C₈) alkyl, or (C₅-C₈) cycloalkyl group, provided that R₃ and R₄ between them contain at least three carbon atoms; or
R₃ and R₄ together with the carbon atoms to which they are attached form a (C₅-C₈) cycloalkene ring; and
each of Y and Z is independently hydrogen or an enzymatically cleavable group selected from the group consisting of
a phosphate group having the formula PO₃W₂ or PO₃V, wherein W is a sugar alcohol, hydrogen or a +1 metal cation and V is a +2 metal cation;
a —C(O)R₅ group, wherein R₅ is C₁₋₂₀ alkyl group; or
an α or β linked sugar residue,
provided that both Y and Z are not hydrogen.

2. The method according to claim 1, wherein the enzymatically cleavable group is an α or β linked sugar residue.

3. The method according to claim 1, wherein the enzymatically cleavable group is a phosphate group having the formula PO₃W₂.

4. The method according to claim 1, wherein R₃ and R₄ together with the carbon atoms to which they are attached form a cyclohexene ring.

5. The method according to claim 1, wherein the esculatin compound is selected from:

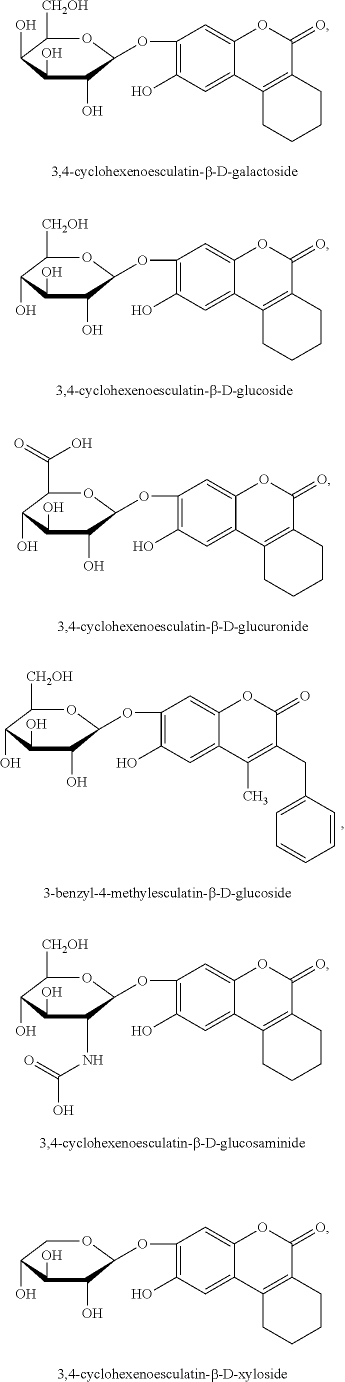

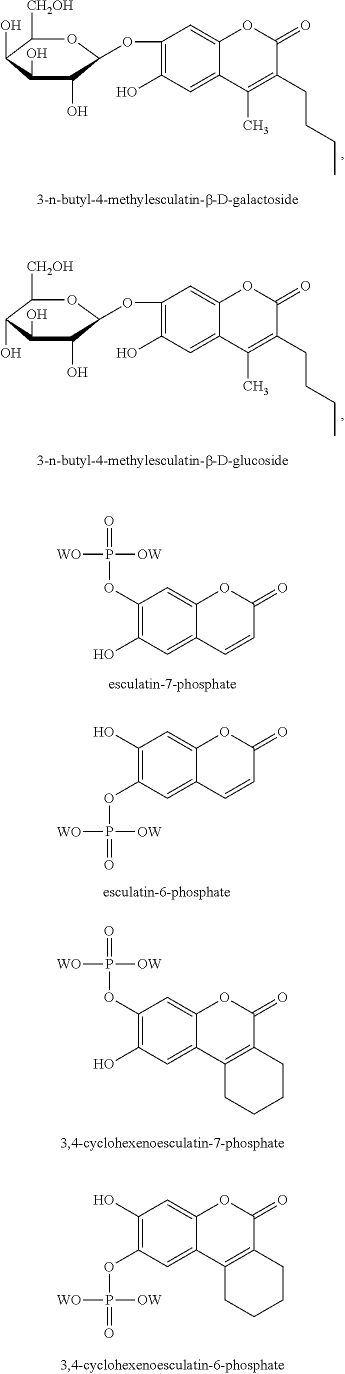

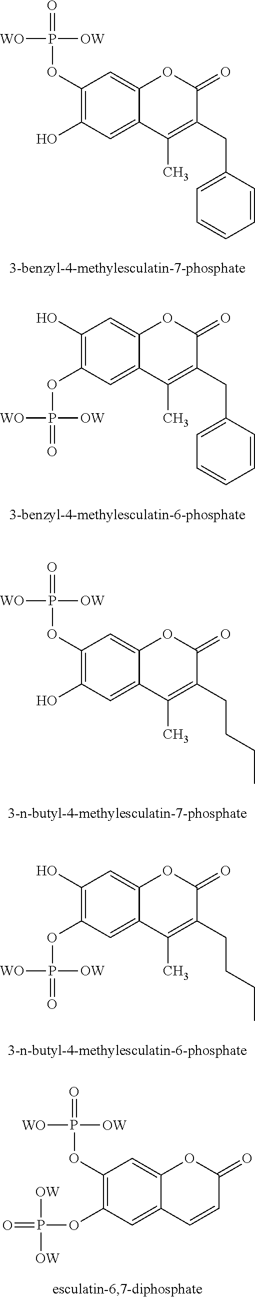

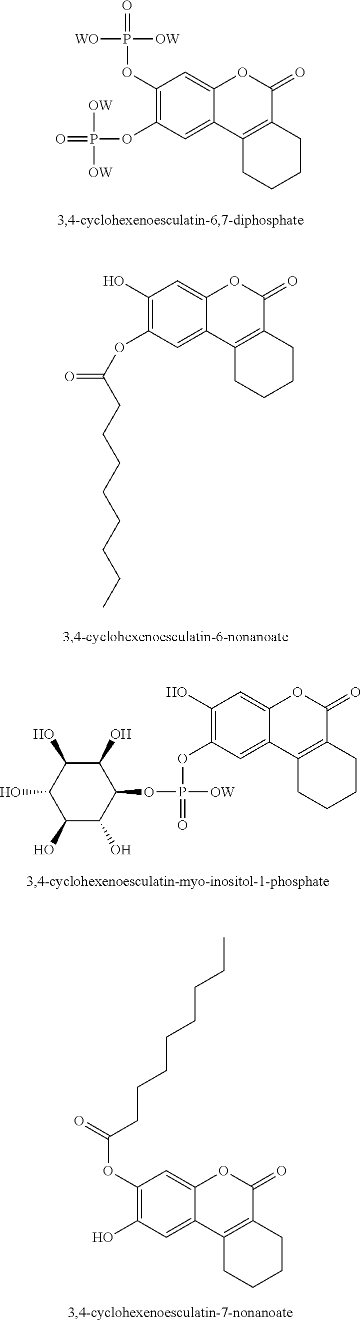

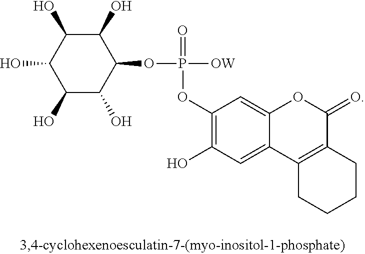

6. The method according to claim 1, wherein the esculatin compound is 3,4-cyclohexenoesculatin-6-phosphate

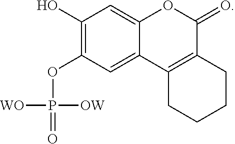

7. The method according to claim 1, wherein the one or more first chromogenic indicator of the enzymatic activity is a pH indicator, a visible color indicator, or a fluorescent indicator.

8. The method according to claim 7, wherein the one or more first chromogenic indicator of the enzymatic activity is 5-bromo-4-chloroindoxyl phosphate.

9. The method according to claim 7, wherein the one or more first chromogenic indicator of the enzymatic activity is 4-methylumbelliferyl-β-D glucopyranoside.

10. The method according to claim 7, wherein the one or more first chromogenic indicator of the enzymatic activity is 4-methylumbelliferyl-β-D glucuronide.

11. The method according to claim 1, wherein the dark precipitate masks the fluorescence or color of the one or more first chromogenic indicator.

12. The method according to claim 1, wherein the presence or absence of the dark precipitate is a secondary or confirmatory test after the one or more first chromogenic indicator.

13. The method according to claim 1, wherein the one or more microorganisms are bacterial microorganisms and are selected from staphylococcus aureus, listeria, salmonella, clostridium, streptococcus, klebsiella, enterobacter, escherichia, citrobacter, proteus, bacillus, pseudomonas, lactobacillus, and coliforms.

14. The method according to claim 13, wherein the staphylococcus aureus is methicillin resistant staphylococcus aureus.

15. The method according to claim 1, wherein the presence of the dark precipitate indicates that the microorganism is not methicillin resistant coagulase negative staphylococcus.

16. The method according to claim 1, wherein the liquid sample is incubated prior to contacting with the esculatin compound.

17. The method according to claim 1, wherein the liquid sample containing the esculatin compound is incubated prior to adding the iron compound.

18. The method according to claim 1, wherein the liquid sample containing the iron compound is incubated.

19. The method according to claim 1, wherein the iron containing compound is ferric ammonium citrate.

20. The method according to claim 1, wherein the method provides further specificity of an enzymatic reaction indicated by the one or more first chromogenic indicator, when the one or more first chromogenic indicator indicates a plurality of possible enzymatic reactions.

21. The method according to claim 1, wherein the one or more first chromogenic indicator of the enzymatic activity is two first chromogenic indicators of the enzymatic activity.

22. The method according to claim 21, wherein two first chromogenic indicators of the enzymatic activity are a colored indicator and a fluorogenic indicator.

23. The method according to claim 22, wherein the colored indicator is 5-Bromo-4-chloro-3-indoxyl nonanoate and the fluorescent indicator is 4-Methylumbelliferyl-β-D-glucuronide.
